(19) United States
(12) Patent Application Publication
(10) Pub. No.: US 2009/0232757 A1
(43) Pub. Date: Sep. 17, 2009

(54) Use of gamma-amino butyric acid as a depigmenting agent

(76) Inventor: Marcel Georges Cohen, Paris (FR)

Correspondence Address: HEDMAN & COSTIGAN P.C., 1185 AVENUE OF THE AMERICAS, NEW YORK, NY, 10036, US

(21) Appl. No.: 12/381,789

(22) Filed: Mar. 17, 2009

Related U.S. Application Data
(63) Continuation of application No. PCT/FR2007/001966, filed on Nov. 29, 2007.

(30) Foreign Application Priority Data
Nov. 30, 2006 (FR) 0610470

(51) Int. Cl. A61K 8/37

(52) U.S. Cl. 424/62

(57) ABSTRACT

Use of gamma aminobutyric acid, one of its salts or one of its lower alkyl ester or a polyol ester as depigmenting or melano modulating agent.

This application is a 371 of PCT/FR2007/001966 filed Nov. 29, 2007.

This invention relates to the necessities of life and more particularly to the body care.

More precisely it has as the subject matter, cosmetic compositions intended to ascertain a whitening of the skin, namely in the cure of esthetic defaults.

More precisely it has as the subject matter, cosmetic compositions intended to insure a whitening of the skin, namely either in the case of esthetic defaults bound to an irregular or excessive pigmentation or intended to insure the whitening of the normal skin allowing to pass from a dark phenotype to a more clear phenotype.

Moreover the compositions in accordance with the inventions find a use in the subjects with clear skin to attenuate the effects of chloasma or ephelids.

This invention has specifically as the subject matter the utilization of gamma aminobutyric acid (GABA), its salts or its derivatives in view of the achievement of depigmenting compositions intented to be applied on the skin and on the teguments.

Gamma aminobutyric acid has already found uses in dermatology, namely as repairing agent for the tissues. Moreover in the International patent application PCT/FR96/01051 the applicant has already disclosed the use of gamma aminobutyric acid and an extract of plants. As a whitening agent this information is very limited and does not provide any precision on the realty of this action. From another side it is not the subject matter of any claim.

The prior art may be illustrated by a number of references citing gamma aminobutyric acid in conjunction with a tension-active agent to insure the transcutaneous passage. Japanese patent JP 09/659141 to Kanebo shows it is an association constituted of pantetheine—S sulfonic acid (or one of its salts), disisopropylamine dichloracetate and of γ-aminobutyric acid or one of its derivatives and a non ionic tensio-active agent having a HLB coefficient of the order of 16, as an example polyoxyéthylène (20) sorbitan monooleate. The content of tension-active agent in the mixture is high (1:10) and is intended to improve the percutaneous absorption of the active ingredient A to facilitate the passage ratio in the horny layer of the skin.

The presence of non ionic tensio-active agent let to presume that the action of the suppressive agent for the production of pigments of melanine-kind is dependant of the transcutaneous absorption of the active ingredient. In fact it has been observes that γ-aminobutyric acid, one of its salt or one of its esters is active by itself while obtaining after a simple application, a significative decrease of intra cellular content of melanine in the cells.

It appears that the present invention relates to distinct compositions acting through a different mechanism and giving rise to different effects.

The prior art also discloses another Japanese patent application 10/194960 to Kanebo. As it appears from the summary in English it relates to a cosmetic composition for the skin evidencing photo protective properties (preventing the skin from getting dark, making dark skin light rapidly). Accordingly it is a preparation the composition of which is clearly distinct: from 0.001 to 5% by weight of at least one kind of product facilitating the blood circulation such as γ-aminobutyric acid [ . . . ] and preferably one agent resulting from a decomposition agent of enzymatic nature obtained from a treatment of an extract derived from rice or a rice tegument with an enzyme such as actinase, pepsine or typsine and thereafter processing to an extraction using a neutral solvent such a purified water.

The nature of rice extract processed with such enzyme and extracted with water is not yet known. It may be suggested that the active component is a mixture of agents which facilitate the circulation or cellular activator and an enzyme decomposition agent (enzyme decomposer) stemming from the rice. It appears this to be a preparation of very distinct composition probably acting while modifying the blood circulation.

Literature also cites a reference Goudzenko Janna Prokovievna (U.S. Pat. No. 5,817,621). It is sure that this reference mentions at column 3, the use of Gabaergic substances and namely the use of L. DOPA and GABA simultaneously. However the compositions the details of which are provided do not relate at all to the use of GABA-ergic substances such as γ-aminobutyric acid but to a complex mixture of Vitamin A, salicylic acid, D-camphor, a GABA-ergic substance, a dopaminergic substance, a cholinolytic substance (atropine), pancreatine, ascorbic acid, pantothenic acid in the form of its calcium salt and vitamin B12.

Such a preparation displays trophic properties and namely restaurs the physiological functionalities of the skin while acting on the gaba-ergic neurones. This patent indicates (column 3, line 53) . . . skin pigmentation becomes normal, the skin becomes rosy end flesh-colored. It is not any matter of depigmentation or of re-pigmentation.

The reference YU (U.S. Pat. No. 6,767,924 B2) constitutes a prior litterature reference as so far it is mentioned aqueous or oily compositions (column 3, lines 37-39 and claim 4). This relates very little to this invention since the cited passage on page 3 mentions the use of such compositions in various pathological conditions of the skin such as spot, lentigine, melasma, blemished skin, and hyperpigmented skin . . . . That means that the compositions disclosed in the cited patent to YU are appropriate for any kind of disturbance in the pigmentation as well as for a bleaching of the skin (blemished skin) or treating a hyperpigmentation of the skin.

It is significant to make clear that the compositions disclosed in the reference YU are made of a mixture of a tensioactive agent having an amphoteric or pseudo amphoteric nature and an alpha hydroxy acid which is polyhydroxylated.

The solution of such hydroxy acids are very acid and the amphoteric agents intervenes for neutralizing this strong acidity and make it compatible with the skin.

This means only that γ-aminobutyric acid cited in this reference of literature, has only a neutralizing potency and cannot be considered as an active ingredient.

It has also been determined hat this acid find a very significant use as a depigmenting agent as compared with the common usual agent such as hydroquinone derivatives (arbutine) or kojic acid while being less cytotoxic . . . .

Gamma aminobutyric acid as well as its salts and derivatives used in such compositions has been studied on isolated cells and namely on cultures of normal human melanocytes. In the used experimental models, it shows an inhibiting activity on tyrosinase and induces a clear decrease of the intracellular content in melanine of the cultured cells. The obtained results are a function of the doses what is in favour of a specific effect of gamma-aminobutyric acid on this kind of parameters.

The compositions according to the invention contains as the active ingredient gamma aminobutyric acid as such or in the form of a salt with a physiologically compatible mineral or organic acid or in the form of a lower alkyl or polyol ester.

Among the salts of gamma-aminobutyric acid which can be used, it may be cited the hydrochloride, the sulphate, the phosphate, the acetate, the propionate, the citrate, the tartarate, the benzoate, the pidolate, the glucose-phosphate, the methane sulfonate, or the p-toluene sulfonate.

The esters of gamma-aminobutyric acid which may be used for the performance of this invention, maybe a methyl, ethyl, propyl, butyl, hexyl ester or a poly ethylene glycol ester, an ester of poly butylene glycol, or an ester of a sugar or of a polyol such as mannitol, sorbitol or glycerol.

Depending on the needs, these esters are those soluble in water or those soluble in the organic solvents such as the oils. This choice will be determined by the nature of the cosmetic composition in which they are incorporated as the active ingredient.

The compositions in accordance with the invention may also contain other active ingredients such as extracts of plants rich in polyphenols or tannins such as white mulberry or α-hydroxy acids such as lactic acid, or even extract of fruits such as lemon or grapefruit, also rich in α-hydroxy acids.

The compositions based on gamma-aminobutyric acid may also be added to thickening agents, gelifying agents, emulsifying agents, flavouring agents, stabilizing agents, preservative agents, and agents which improve the feeling or the fluidity.

### BRIEF DESCRIPTION OF THE DRAWINGS

FIG. 1 is a graph of the cytotoxicity of the active ingredients vs normal human melanocytes cultivated in mono-layers of Example 3.

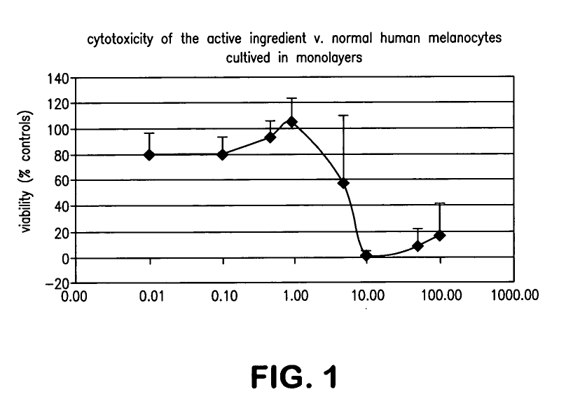

FIG. 2 is a graph of cytotoxicity of lactic acid vs normal human melanocytes cultivated in mono-layers of Table II.

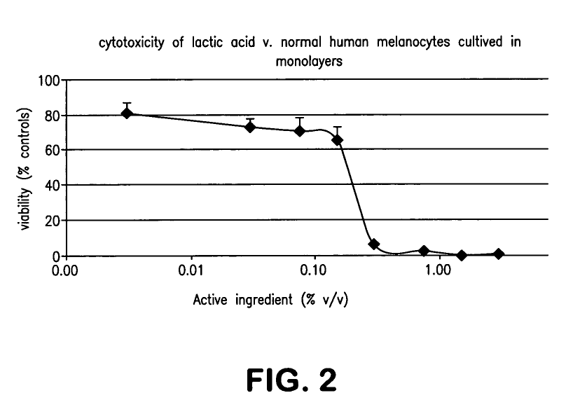

FIG. 3 is a graph of cytotoxicity of GABA vs normal human melanocytes cultivated in monolayers of Table III.

FIG. 4 is a graph of synthesis of melanine in the presence of complex in a pattern of human melanocytes in mono-layers cultures.

It may be cited in this respect as emulsifying agent polyethylene glycol stearate or esters of sugars such as sucrose esters.

As a thickening agent it may be cited the derivatives of cellulose such as methylcellulose, ethylcellulose, β-hydroxyethylcellulose, carboxymethylcellulose, or a cross-linked carboxymethylcellulose such as ACDISOL®.

A gelifying agent will be for example a polymer of acrylic acid or of acrylamide such as the carbomers sold under the Trade Name CARBOPOL® (GOODRICH).

The content into gamma aminobutyric acid in the compositions may range from 0.4 to 10% of the whole composition with a preference for 0.5 to 5%.

The examples which follow illustrate the invention without anyhow limit it.

### EXAMPLE I

### Cream Based on Gamma-Aminobutyric Acid as the Hydrochloride

Gamma aminobutyric acid hydrochloride 7.5 g

White mulberry extract 0.5 g

Lemon extract BG 2.0 g

Cire Lanette® 3.75 g

Emulsifying agent 0.5 g

Water enough for 100 g

### EXAMPLE II

### Gel Based on Gamma Aminobutyric Acid

Gamma aminobutyric acid 2 g

Propyleneglycol 5 g

Sweet almond oil 10 g

Oil of silicon 0.1 g

Germaben II 0.02 g

Carbopol® 936 0.4 g

Triethanolamine 0.1 g

Perfume of flowers enough

Purified water 80 g

### EXAMPLE III

### Study on the Cytotoxicity of the Compositions Based on Gamma Aminobutyric Acid

The aim of this study has been to evaluate the cytotoxicity of gamma-aminobutyric acid, that of the white mulberry extract and that of the lemon extract separately taken in relation to that of a well-known α-hydroxy acid, such as lactic acid (compound E).

The determination is performed with normal human melanocytes obtained from a young patient (4 years old). For the performance of the tests, the cells have been cultured until obtention of confluent mono-layers.

### Incubation of the Cells:

The cells have been incubated for 72 hours in the absence (controls) or in the presence of increasing concentrations of compound E or of each of the tested compounds. For compound E the following concentrations have been tested 0.003; 0.03; 0.075; 0.15; 0.5; 0.75; 1.5 and 3% (v/v) and for the product according to the invention: 0.01; 0.1; 0.25; 0.5; 1; 2.5; 5; 10% (v/v).

The tested compound has been prepared by directly diluting gamma-aminobutyric acid in ultra pure water in an amount enough for 100% (V/V).

As a comparison one has prepared a 10% solution of kojic acid and a 30% solution of lactic acid (composition E).

The tested preparations have been then directly diluted in the incubation medium for the melanocytes.

### Evaluation of the Effects:

At the end of the incubation period, the viability of the cells has been evaluated using a spectrophotometric method for dosing the activity of the intracellular phosphatases. According to the method of Yan T. et al. (Anal. Biochem. 24 (1996) 103-108), one doses the p-nitrophenol resulting from the conversion of the phosphate of p-nitrophenol by means of the intracellular phophatases of the viable cells present in the wall of culture.

### RESULTS

Lactic acid (composition E) after 72 H incubation significatively decreases the number of viable melanocytes present in the walls of culture. This effect may be detected (less than 80% of the viable cells still present in the walls of culture) from the doses of 0.03%. Lactic acid is then clearly cytotoxic.

Gamma-aminobutyric acid display any detectible cytotoxic effect under the same conditions only from a dosis of 0.5%. As a consequence the depigmenting composition according to the invention which contains gamma-aminobutyric acid at 10% shows cytostatic effect only at higher experimental doses (see fig. I, II and III and Table I).

TABLE I
Study on the cytotoxicity of lactic acid (E) and the composition
based of gamma aminobutyric acid.
Active ingredient (E)

| | Active ingredient E | | | | | | | |
|---|---|---|---|---|---|---|---|---|
| | (%, v/v) | | | | | | | |
| | 0.003 | 0.03 | 0.075 | 0.15 | 0.3 | 0.75 | 1.5 | 3 |
| Percentage of | 87.7 | 78.4 | 75.6 | 74.3 | 7.0 | 2.7 | 0.2 | 1.4 |
| viability | 76.0 | 70.0 | 74.9 | 62.5 | 6.7 | 2.5 | 0.6 | 0.8 |
| (vs controls) | 78.8 | 71.3 | 61.9 | 60.9 | 4.9 | 1.9 | −0.3 | 0.3 |
| Mean | 80.8 | 73.2 | 70.8 | 65.9 | 6.2 | 2.4 | 0.2 | 0.8 |
| s.d. | 6.1 | 4.5 | 7.7 | 7.3 | 1.1 | 0.4 | 0.4 | 0.5 |

Composition according to the invention

| | Composition | | | | | | | |
|---|---|---|---|---|---|---|---|---|
| | (%, v/v) | | | | | | | |
| | 0.01 | 0.1 | 0.25 | 0.5 | 1 | 2.5 | 5 | 10 |
| Percentage of | 96.6 | 88.0 | 68.0 | 60.6 | 8.2 | 1.8 | 0.2 | 0.1 |
| viability | 109.7 | 88.6 | 88.1 | 78.7 | 8.3 | 0.5 | −0.2 | −0.1 |
| (vs controls) | 111.7 | 101.6 | 96.1 | 89.5 | 10.7 | 1.6 | −0.4 | 0.3 |
| Mean | 106.1 | 92.7 | 84.1 | 76.3 | 9.1 | 1.3 | −0.1 | 0.1 |
| s.d. | 8.2 | 7.7 | 14.5 | 14.6 | 1.4 | 0.7 | 0.3 | 0.2 |

The compositions based on white mulberry extract and lemon extract have also moderate effects on cytotoxicity.

### EXAMPLE IV

“Melanomodulating” effect of the active ingredients contained in the compositions according to the invention.

The search of a “melanomodulating” effect has been performed in two steps:
melanomodulating effect of the active ingredients taken separately at predetermined concentrations in the cytotoxicity tests.
Melanomodulating effect of the complex, i.e. GABA in admixture with the other ingredients, white mulberry, lemon and lactic acid.

### Step 1

These tests have been performed on a model of normal human melanocytes cultured on monolayer cultures.

Gamma aminobutyric acid, lemon extract and white mulberry extract have been solubilized and directly diluted in the culture medium.

Incubation of the Cells with the Tested Products:

The normal human melanocytes have been incubated for 72 hours in the absence (controls) or in the presence of a reference product (Kojic acid 250 μM) or of increasing concentrations of the tested active ingredients.

Gamma aminobutyric acid (composition A) 0.001; 0.01; 0.1%

White mulberry extract (composition C) 0.01; 0.1; 0.5%

Lemon extract (composition D) 0.01; 0.1; 1%

In this method the ratio melanine/proteins is determined as a percentage of the controls. Also it has been determined the obtained values with the solvent alone and with a 250 μM solution of Kojic acid in this solvent (DMSO) taken as reference substance.

Table II provides the obtained results with the active ingredients taken separately A (gamma aminobutyric acid), C, D and lactic acid (composition E). Gamma aminobutyric acid provides statistically significant results from a concentration of 0.1%. Compounds C and D have only moderate “melanomodulating” effect.

### Step 2

The effect of a complex containing the active ingredients A (gamma aminobutyric acid), C, D and lactic acid (composition E) has also been evaluated as a comparating element, on the synthesis of melanine in a model of normal human melanocytes cultured in a monolayer culture.

TABLE II
Vehicle (DMSO) and reference product

| | | |
|---|---|---|
| | | Kojic Acid |
| Controls | DMSO | 250 μM |

| | | | | | | |
|---|---|---|---|---|---|---|
| Ratio | 96.8 | 100.6 | 95.6 | 98.2 | 73.1 | 65.9 |
| Melanine/ | 97.8 | 110.7 | 98.4 | 105.4 | 84.3 | 69.5 |
| Proteins | 93.0 | 91.4 | 99.4 | 105.9 | 84.5 | 78.1 |
| (% of the | 110.5 | 99.2 | 95.7 | 97.3 | 93.9 | 66.3 |
| controls) | 100.6 | 106.4 | 97.2 | 91.5 | 88.0 | 70.6 |
| | 102.4 | 102.9 | — | — | — | — |
| | 100.9 | 99.3 | — | — | — | — |
| | 96.1 | 91.4 | — | — | — | — |

| | | | |
|---|---|---|---|
| Mean | 100.00 | 98.45 | 77.40* |
| s.d. | 4.56 | 3.33 | 4.57 |

*significatively different mean from that of the controls group “medium
alone” (p < 0.05).

Active ingredients tested

| | A (%, w/v) | | | C (%, v/v) | | | D (%, v/v) | | |
|---|---|---|---|---|---|---|---|---|---|
| | 0.001 | 0.01 | 0.1 | 0.01 | 0.1 | 0.5 | 0.01 | 0.1 | 1 |
| Ratio | 98.8 | 87.0 | 74.3 | 104.8 | 138.2 | 162.2 | 94.9 | 105.5 | 133.9 |
| Melanine | 108.6 | 101.8 | 76.4 | 106.8 | 147.6 | 160.4 | 104.2 | 94.8 | 134.8 |
| Proteins | 98.3 | 101.1 | 74.2 | 99.8 | 154.3 | 155.9 | 98.0 | 104.4 | 130.5 |
| (% of the | — | — | 71.3 | — | — | 165.5 | 95.7 | 97.3 | 142.3 |
| controls | — | — | — | — | — | 181.0 | 97.2 | 91.5 | 122.7 |
| | — | — | — | — | — | 181.1 | — | — | 133.5 |
| | | | | | | 177.2 | — | — | — |
| | — | — | — | — | — | 178.2 | — | — | — |
| Mean | 101.9 | 96.6 | 74.0* | 103.8 | 146.7 | 170.2* | 99.0 | 101.6 | 133.0* |
| s.d. | 5.8 | 8.4 | 2.1 | 3.6 | 8.1 | 10.3 | 4.8 | 5.9 | 6.4 |

*significatively different mean from that of the controls “medium alone” (p < 0.05).
A: composition based on gamma aminobutyric acid
C: composition based on white mulberry
D: composition based on lemon extract

### Tested Products:

The compositions have been prepared by directly diluting gamma aminobutyric acid (A), active ingredients C and D and lactic acid (compound E) in ultrapure water at the following concentrations:

White mulberry extract 10%

Lemon extract 1%

Lactic acid 1.5%

Ultrapure water

The tested compositions have therafter been directly diluted in the incubation medium for the melanocytes at a concentration of 0.1, 0.5 and 1% (v/v).

### Testing System:

Normal human melanocytes have been utilized stemming from a young 4 years old patient. They have been cultured in a monolayer culture until of a confluence of 80%.

### Reference Product:

It has been used as reference Tyrosinase inhibitor, Kojic acid at 250 μM.

### Incubation of the Cells:

The melanocytes have been incubated for 72 hours at 37° C. under wet atmosphere and under 5% CO₂ in the absence (controls) or in the presence of Kojic acid or increasing concentrations of the tested active ingredients (0.1, 0.5, 1%).

### Evaluation of the Anitimelanine Effects:

1—Dosage of Melanine:
at the end of the incubation period, the intra cellular content of melanine has been quantified after cellular lysis, using a spectrophotometric measure at 405 nm.

2—Dosage of the Proteins:
at the end of incubation period the proteins contained in the cellular lysates have been determined using a spectrocolometric method based on Coomassie Blue according to the method of Bradford M. (Anal. Biochem. 72 (1976) 248-254).

3—Results:
the results have been given as a percentage of melanine v. controls (mean+/−standard deviation SD) calculated from the obtained values in μg of intracellular melanine per mg of total proteins of the cellular layer.

The statistical significativity of the observed differences between the conditions of the “controls” and of the “tested products” has been determined in accordance with a variance analysis using only one factor (ANOVA) followed by a Holm-Sidak test (*=p<0.05).

The obtained results with the composition based on gamma aminobutyric acid evidence a significative decrease of the intra cellular content of melanine in the cultured cells.

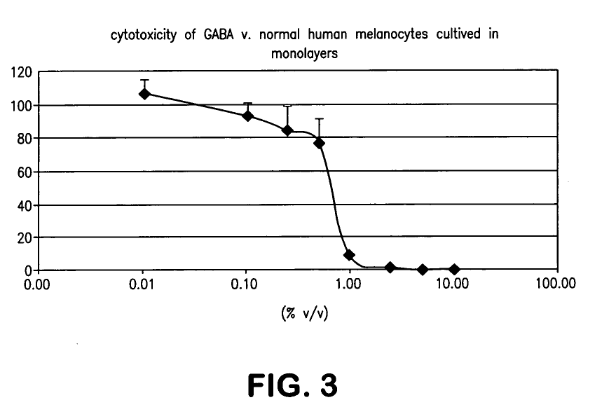

The obtained value with 0.1% of active ingredient is not statistically significative. The obtained values with a 0.5% concentration (−19.7%) and a 1% concentration (−25.4%) are statistically significative (p<0.05). These results are shown in FIG. 3 and in the Table III.

Kojic acid at 250 μM used as a reference melanogenesis inhibitor, as a comparison substance, produces a significative decrease of the intra cellular content of melanine in the culture cells (decrease of 15.5%) (p<0.05).

This result shown in Table III evidences the proof of the validity of the investigation method.

The obtained results with the compositions based on gamma aminobutyric acid are dosis-dependent what is in favour of a specific effect of this complex on the studied parameter.

### RESULTS

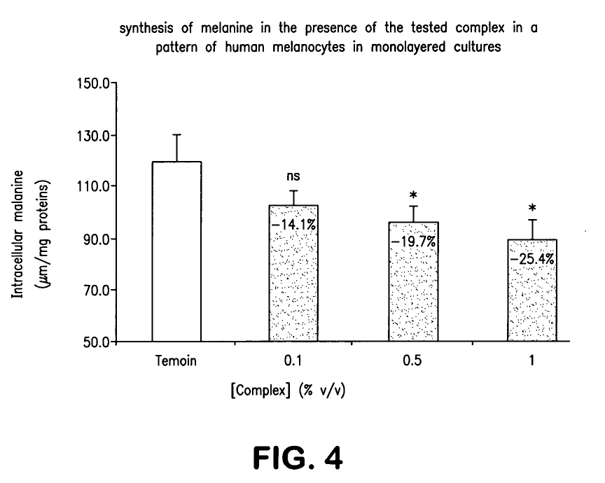

The results shown in FIG. 4 and in the Table III hereabove feature that in the retained experimental conditions the tested complex significatively reduces the intra cellular content of melanine in the cultured cells:

Complex at 0.1% (v/v)→−14.1% (ns)

Complex at 0.5% (v/v)→−19.7% (p<0.05)

Complex at 1% (v/v)→−25.4% (p<0.05)

TABLE III
Tested complexes:

| | | Tested complex (% v/v) | | |
|---|---|---|---|---|
| | Controls | 0.1 | 0.5 | 1 |
| Ratio | 131.4 | 108.1 | 98.4 | 97.7 |
| melanine/ | 115.1 | 102.4 | 100.1 | 83.3 |
| proteins (% | 111.1 | 96.6 | 88.8 | 85.8 |
| of the | 103.8 | 103.6 | 88.6 | 92.2 |
| controls) | 104.5 | — | — | — |
| Mean | 119.2 | 102.4 | 95.8* | 88.9* |
| s.d. | 10.8 | 5.8 | 6.1 | 7.7 |
| % of the | 100 | 85.9 | 80.3 | 74.6 |
| controls | | | | |

*statistically different mean from the controls group “medium alone”
(p < 0.05).

Reference product: Kojic Acid at 250 μM

| | Controls | Kojic Acid 250 μM |
|---|---|---|
| Ratio | 131.4 | 106.3 |
| melanine/ | 115.1 | 93.2 |
| Proteins (% | 111.1 | 102.8 |
| of the | 103.8 | 94.1 |
| controls) | 104.5 | 97.9 |
| Mean | 119.2 | 100.8* |
| s.d. | 10.8 | 6.8 |
| % of the | 100 | 84.5 |
| controls | | |

*significatively different means from that of the controls group “medium
alone” (p < 0.05).

Gamma aminobutyric acid and its salts with a mineral or organic acid and its derivatives are utilized in the form of aqueous or oily cosmetic compositions selected among milks, lotions, oil in water emulsions, or water in oil emulsions, creams, gels and toilet waters.

Concentrations in gamma aminobutyric acid may vary within broad proportions ranging from 0.4 to 10% by weight of the total compositions. A more preferred concentration ranges from 0.5 to 5% by weight.

When gamma aminobutyric is used in the form of a salt or a derivative the utilized amounts are calculated taking into account of the content of gamma aminobutyric acid in the salt or in the derivative. In the case of an ester of a polyphenol, the concentrations into gamma aminobutyric acid have to be calculated to achieve a content into active ingredient ranging from 0.4 to 10% by weight in the cosmetic compositions according to the invention.

1-12. (canceled)

13. A method of whitening the human skin comprising applying to a human skin a whitening composition comprising an effective amount of a member of the group consisting of gamma aminobutyric acid, a salt thereof, a lower alkyl ester thereof and a polyol ester thereof.

14. The method of claim 13 using free gamma aminobutyric acid.

15. The method of claim 13 using a salt of gamma aminobutyric acid.

16. The method of claim 13 using the salt of gamma aminobutyric is with a physiologically compatible mineral or organic acid.

17. The method of claim 16 wherein the salt is the hydrochloride of gamma aminobutyric acid.

18. The method of claim 13 using a lower alkyl ester of gamma aminobutyric acid.

19. The method of claim 18 wherein the lower alkyl ester is dissolved in water or an organic solvent.

20. The method of claim 13 using gamma aminobutyric acid is applied in a vegetal extract rich in polyphenols or in tannins.

21. The method of claim 13 wherein the whitening composition is a topic cosmetic composition.

22. The method of claim 21 wherein the whitening composition is an aqueous or oily composition selected from the group consisting of milks, lotions, oil in water emulsion, water in oil emulsion, creams, gels and toilet water.

23. The method of claim 13 wherein the active ingredient is at a concentration of 0.4 to 10% by weight of the composition.

24. The method of claim 23 wherein the concentration is 0.5 to 5% by weight.
